(19) United States
(12) Patent Application Publication
(10) Pub. No.: US 2006/0184104 A1
(43) Pub. Date: Aug. 17, 2006

(54) Needle guard

(75) Inventors: Paul S. Cheney II, Winnetka, CA (US); Sheldon B. Moberg, Thousand Oaks, CA (US); Julian D. Kavazov, Arcadia, CA (US); Susie E. Maule, Pasadena, CA (US); Arin N. Holecek, Lakewood, CA (US); Christopher G. Griffin, Sylmar, CA (US); Mark D. Holt, Moorpark, CA (US)

Correspondence Address: PILLSBURY WINTHROP SHAW PITTMAN LLP, P.O BOX 10500, McLean, VA, 22102, US

(73) Assignee: Medtronic Minimed, Inc.

(21) Appl. No.: 11/058,074

(22) Filed: Feb. 15, 2005

(51) Int. Cl. A61M 31/00

(52) U.S. Cl. 604/93.01

(57) ABSTRACT

A needle guard for an insertion device generally used with an infusion set. The needle of the insertion device is commonly adapted for puncturing at one end and including at the opposite end a hub. The needle guard includes an opening adapted to receive the insertion device and a locking mechanism adapted to secure the insertion device in the needle guard. By pushing the needle hub into the needle guard for disposal, barbs on the needle hub will mate with undercuts on the inside of the needle guard and secure the needle hub in a position where the needle is covered, preventing unintended contact with the needle.

### BACKGROUND OF THE INVENTION

1. Field of Invention

Embodiments of the present invention relate generally to an insertion device used commonly for subcutaneously puncturing a site in a patient to insert a soft cannula that forms part of an infusion set for subcutaneous delivery of a fluid, drug or other infusion. More particularly, embodiments of the present invention relate to a needle guard capable of preventing the risks of unintended harmful injuries from the needle.

2. Description of Related Art

Subcutaneous infusion sets are used for delivering a fluid, medication or other infusion to an infusion site in a patient. These devices commonly include a delivery tube connected to an infusion pump or other fluid or medication delivering device. Such an infusion set may include a base portion with a cannula inserted in the skin of a patient. The cannula is inserted into the skin of a patient with the aid of an insertion device. The cannula may be held in place at the infusion site with the aid of an adhesive attached at the base of the cannula housing. This allows for the possibility of disconnecting the infusion set from other parts of the infusion system in order to improve user comfort.

The use of a cannula further improves patient comfort. The cannula is more flexible than a rigid needle and allows the user to perform activities with much less pain or discomfort. While this allows for increased mobility, there remain disadvantages to patients for other reasons. To insert the cannula in place, an insertion device, commonly including a needle, is used and then discarded after attaching the cannula to the infusion site. Therefore, upon withdrawal of the needle hub from the cannula, there are potential risks of unintended harm from the exposed end of the needle.

Because of the risk of harm during or after disposal of the needle, it is helpful if the needle can be quickly and securely covered. In some cases, the patient may not have easy access to a sharps container and therefore will have to carry the insertion device and needle in their pocket, purse, bag, etc. until a sharps container or other suitable container is available. Thus, the coverage should withstand the entire process and remain secured after it reaches its final disposal location.

Therefore, it would be useful to provide a safe and convenient needle guard with which the needle of an insertion device may be easily covered in its shipped condition, protecting the needle from damage during shipping, and after use, reducing the danger of exposing persons to an exposed needle. An insertion device with a needle guard is described in U.S. Pat. No. 6,355,021. The insertion device comprises a needle, cannula housing, adhesive patch and needle guard. The needle guard includes a shield that can be manipulated to cover the needle as well as secure the entire insertion device.

However, while some previously known needle guards have provided ways to prevent unintended and harmful needle pricks, there are common disadvantages. First, the other known needle guards often utilize small and difficult to handle pieces for shielding the needle. Additionally, these needle guards generally require the user to fit the pieces together in some form of complicated maneuvering to secure the needle. The cumbersome assembly process takes additional time and space, and the assembly process itself presents additional dangers of causing needle pricks.

### BRIEF SUMMARY

In accordance with an embodiment of the invention, a-needle guard has been developed that can be used with insertion devices commonly associated with an external infusion system. For example, insertion devices that may be covered by the needle guard of the invention may be those such as are described in U.S. Pat. Nos. 6,520,938 and 6,056,718, which are herein incorporated by reference. Another example is described in co-pending U.S. application Ser. No. 11/003225, entitled Medication Infusion Set, filed on Dec. 3, 2004, which is herein incorporated by reference. The needle guard provides a cover for the needle before and after use with an infusion set. The needle guard may accompany the insertion device as packaged to the user, ensuring that the needle will not cause any harm in delivery to the user and also serve to protect the needle during shipping. An embodiment of the present invention includes an entire assembly in which the needle guard is delivered with a needle hub that is connected to a cannula housing. Some infusion sets include an adhesive patch that is attached to the base of the cannula housing. The adhesive patch may be covered with a one or more piece liner that the patient removes to expose the adhesive side. The liner keeps the adhesive sterile and protects the adhesive from being removed or from attaching to anything else until the patient wishes to use the infusion set. Thus, the assembly may further include the cannula housing with the adhesive patch and liner.

Additionally, the needle guard can be used to cover the needle after use. Generally, after the placement of the cannula and infusion set at the infusion site, the needle hub is removed and the infusant may be delivered through a connector. The needle guard is subsequently used to cover and secure the needle on the insertion device so that the sharp end of the needle is not exposed. This allows the needle to be safely disposed.

In one embodiment, the needle guard includes a thin body that is of a wide lateral shape with an opening that can slip over the needle of an insertion device like a sheath. The needle guard may be configured as a single piece, but the single piece is not required. In another embodiment the body may include a top layer and a bottom layer that are connected at one end. The layers may be angled away from each other on an end opposite to the end of attachment to form an opening between the top layer and bottom layer. The needle of the insertion device to be covered is received by the opening. Inside the end of the needle guard, away from the opening, there may be a needle tip groove. The needle tip may be held in the groove after the needle guard is secured over the needle hub.

When the needle guard is fit over the needle hub, the interaction of undercuts on the inner side of the needle guard secures the needle guard to the needle hub. The undercuts in the needle guard are adapted to mate with corresponding barbs on either side of the needle hub. The barbs can initially be held in by the cannula housing to prevent interaction with the undercuts, as in when the assembly is being delivered to the user. After the cannula housing is displaced at the infusion site, the needle hub is removed from the cannula housing, exposing the barbs. When the needle hub is re-inserted into the needle guard without the cannula housing, the barbs and undercuts are adapted to secure the needle guard to the needle hub. Various embodiments of the locking mechanisms may be used to secure the needle guard in a covered position whereby the needle guard is secured over the needle hub of the insertion device and covers the needle, such as clips, hooks, or other connecting pieces. The locking mechanism may hold a specific position either temporarily or permanently.

The needle guard may also include fingers extending outward from the opening. These fingers further include barbs on the tips of each finger, called finger barbs. The finger barbs are adapted to mate to the back end of the needle hub. The fingers additionally correspond with holes on the needle hub. The barbs on the tips of each finger can extend to and mate into the hub holes, but may also be released by disconnecting the barbs from the hub holes. The fingers and holes are useful in providing a covered position in which the whole assembly, including the needle guard and needle hub connected to a cannula housing, can be delivered safely. When the needle hub is covered by the needle guard without the cannula housing, the fingers can extend farther past the hub holes so that they do not interact with the hub holes.

The needle guard may be made from a suitable plastics material that will allow it to flex and bend, such as polypropylene. However, the needle guard may also be made out of a non-flexible material, such as polycarbonate, if preferred. Alternatively, the needle guard may be composed of any suitable flexible or non-flexible material such as polyethylene, polyurethane, polyvinyl chloride, resins, polymers, ceramics, composites, or the like. The body may have various shape embodiments that facilitate convenient handling. For example, an embodiment that includes a wide-shaped body makes it possible to easily hold or grab the needle guard when removing from or covering the needle. The shape may also be preventative of slips during attempts to use the needle guard. The body of the needle guard may also closely fit the insertion device to avoid taking up excess space. Once the needle guard is fitted over the needle hub, the barbs engage to help the needle guard stay securely on the insertion device and should stay secure during shipping, handling and aging.

### BRIEF DESCRIPTION OF THE DRAWINGS

A detailed description of embodiments of the invention will be made with reference to the accompanying drawings, wherein like numerals designate corresponding parts in the figures.

FIG. 1 is a perspective view showing an external configuration of a needle guard mounted on an insertion device with a cannula housing in a covered position according to an embodiment of the invention.

FIG. 2 is a horizontal partially transparent view of a needle guard mounted on an insertion device with a cannula housing in a covered position according to an embodiment of the invention.

FIG. 3 is a side view of a needle guard mounted on an insertion device with a cannula housing in a covered position according to an embodiment of the invention.

FIG. 4 is a perspective view showing an external configuration of a needle guard mounted on a needle hub without a cannula housing in a covered position according to an embodiment of the invention.

FIG. 5 is a horizontal cross-sectional view of a needle guard mounted on a needle hub without a cannula housing in a covered position according to an embodiment of the invention.

FIG. 6 is a side view of a needle guard before and after the barbs are mated according to an embodiment of the invention.

FIG. 7 is a perspective view of a needle guard with an insertion device in a covered position and a needle guard with a needle hub, showing the manner in which the needle is brought to the covered position, according to an embodiment of the invention.

FIG. 8 is a perspective view of a needle guard with an insertion device in a covered and uncovered position according to an embodiment of the invention.

FIG. 9 is a perspective view of a needle guard in an uncovered and covered position according to an embodiment of the invention.

FIG. 10 is a perspective view of a needle guard in a covered position with a cannula housing, in an uncovered position, and in a covered position without the cannula housing according to an embodiment of the invention.

### DETAILED DESCRIPTION

In the following description, reference is made to the accompanying drawings which form a part hereof and which illustrate several embodiments of the present invention. It is understood that other embodiments may be utilized and structural and operational changes may be made without departure from the scope of the present invention.

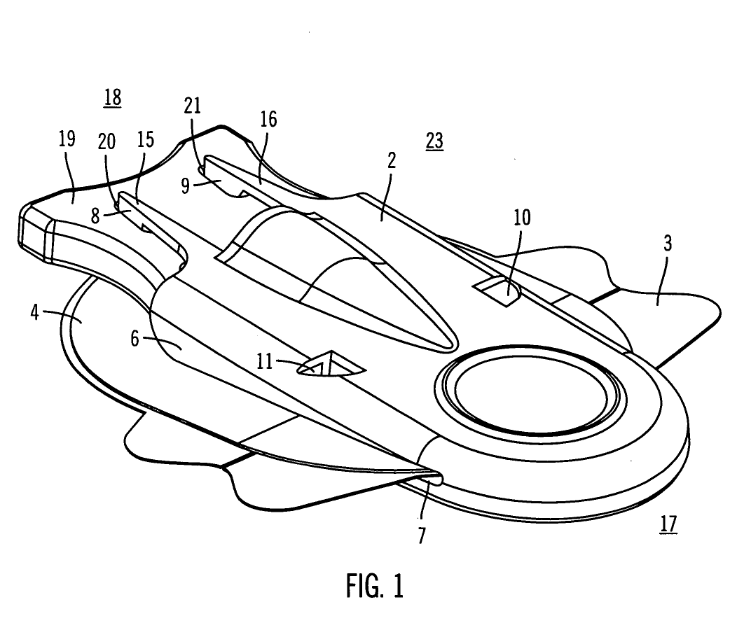

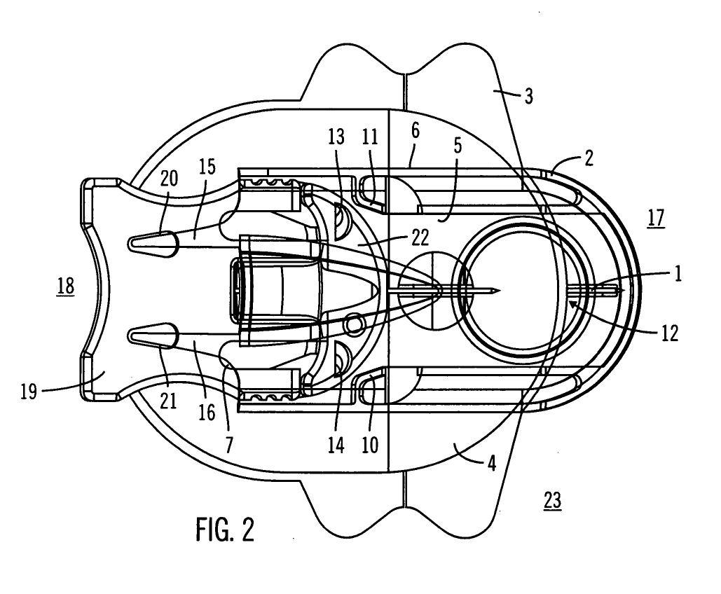

As shown in FIG. 1, one embodiment of the needle guard 17 comprises a body 2 of a wide lateral shape that closely fits the insertion device 18. The needle guard 17 includes an opening 7 that can slip over the needle 1 of an insertion device 18. A locking mechanism may include undercuts 10 and 11 within the body 2 and fingers 15 and 16 with finger barbs 8 and 9 extended from the backside of the body 2. The locking may be engaged when the needle hub 19 is covered by the needle guard 17. The locking may be temporary or permanent. The thin, wide-shape of the body 2 facilitates convenient handling, however, the body may be thicker or narrower as long as its opening can receive the needle and insertion device. The shape makes the needle guard 17 easy to hold or grab when using it to remove or cover the insertion device 18. In other alternatives, the body may have alternative body structures, such as being ridged, having an hour glass shape or other structures to effectuate handling. The body 2 has an inner side 5 (see FIG. 2) and an outer side 6 with an opening 7 in the body 2 adapted to receive the insertion device 18. The needle guard 17 may be delivered to the user as an entire assembly 23 (see FIG. 2), including the needle guard 17 and the insertion device 18 with a cannula housing 22 and attached adhesive patch 4 and liners 3. Some infusion sets include an adhesive patch that is attached to the base of a cannula housing. The adhesive may be covered with a one or more piece liner system that the patient removes to release the adhesive side. The liners 3 keep the adhesive sterile and protect the adhesive from being removed or from attaching to anything else until the patient wishes to use the infusion set.

In FIGS. 1, 2, and 3, a covered position of the needle guard 17 is shown. The insertion device 18 is secured within the opening 7 of the body 2. The whole assembly 23, including the needle guard 17 and the insertion device 18 connected to the cannula housing 22 with the adhesive patch 4 and liners 3, can be sent to the user in this manner to provide a safe delivery in which the needle I of the insertion device 18 is covered while in transit. In this covered position the insertion device 18 is connected to the cannula housing 22 and may be ready for delivery. The needle guard 17 may be secured by the interaction of undercuts 10 and 11 on the inner side 5 of the needle guard when the needle guard 17 is fit over the needle hub 19 of the insertion device 18. The undercuts 10 and 11 in the needle guard 17 are adapted to mate with corresponding barbs 13 and 14 on either side of the needle hub 19 of the insertion device 18. In this covered position, the barbs 13 and 14 can be held in by the cannula housing 22 to prevent interaction with the undercuts 10 and 11, as in when the assembly 23 is being delivered to the user. This allows the insertion device 18 to be removable from the needle guard 17 when ready for use. In another alternative, a cap, shield or separate guard of some sort may be included to hold the barbs in rather than the cannula housing. In this manner, the insertion device and needle guard can be packaged together without the cannula housing. The cap can be included to hold in the barbs to prevent mating with the undercuts. The needle guard can be used to releasably cover the needle of the insertion device while being delivered to the purchaser.

In the covered position, the finger barbs 8 and 9 are adapted to mate to the back end of the needle hub 19. The fingers 15 and 16 additionally correspond with holes 20 and 21 on the needle hub 19. The fingers 15 and 16 of the needle guard 17 extend to the hub holes 20 and 21 and are mated to the corresponding hub holes 20 and 21 by barbs 8 and 9 on the fingers 15 and 16. The fingers 15 and 16 can be released by removing the finger barbs 8 and 9 from the hub holes 20 and 21.

The finger barbs 8 and 9 and hub holes 20 and 21 are useful in providing a covered position in which the whole assembly 23, including the needle guard 17 and insertion device 18 connected to a cannula housing 22, can be delivered safely before the needle needs to be exposed for insertion. In other alternatives, the number of fingers, finger barbs, and hub holes may be varied. For example, one alternative locking structure may have one finger with a barb to mate to one hub hole on the needle hub. Additionally, the fingers may use alternative locking structures to barbs, such as such as hooks, clips, or other connecting pieces.

The partially transparent view shown in FIG. 2 more clearly illustrates the manner in which the insertion device 18 is covered by the needle guard 17. The cannula housing 22, with the attached adhesive patch 4 and liner 3, is shown holding in the barbs 13 and 14 of the needle hub 19. The cannula housing 22 prevents the mating of the barbs 13 and 14 with the undercuts 10 and 11 in the needle guard 17. The barbs 8 and 9 of the fingers 15 and 16 are mated to the hub holes 20 and 21, securing the insertion device 18 within the needle guard 17 for safe delivery and handling until use. The needle 1 is covered by the body 2 of the needle guard 17.

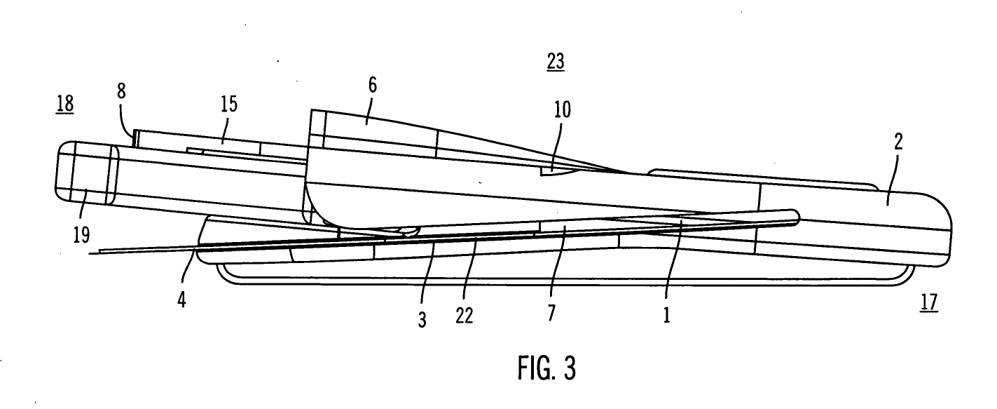

A side external view of the needle guard 17 is shown in FIG. 3 as part of a whole assembly 23, including the insertion device 18 connected to the cannula housing 22 with adhesive patch 4 and liners 3. The needle guard is releasably secured to the insertion device 18 by the finger barbs 8 and 9. The finger barbs 8 and 9 are able to mate to the holes 20 and 21 on the needle hub 19. The needle 1 is temporarily secured within the needle tip groove (not shown) to prevent unintended contact with the needle 1.

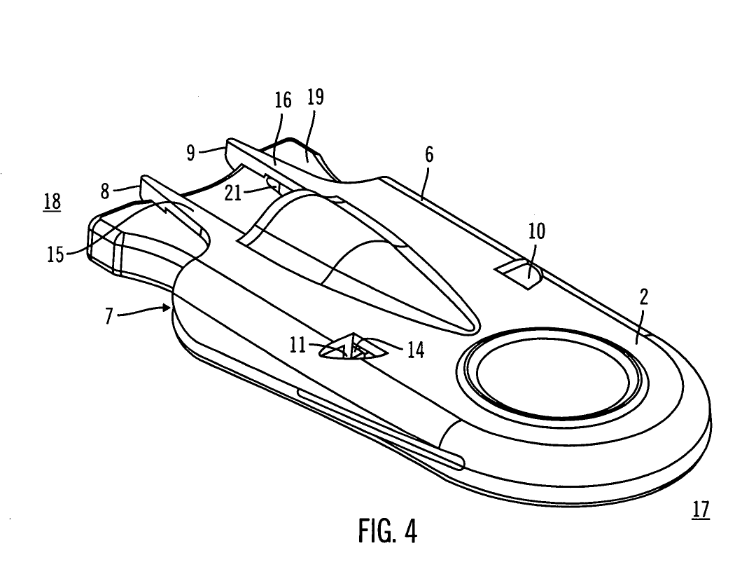

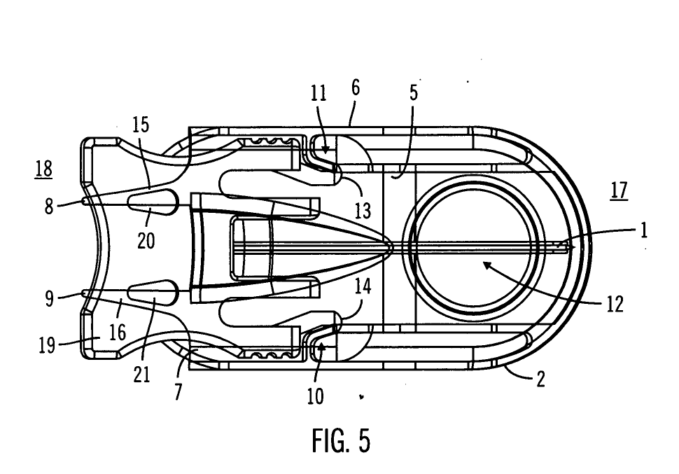

In FIGS. 4 and 5 an alternative of the covered position is illustrated. This position may also be temporary or permanent. After the needle guard is removed for insertion, the insertion device facilitates the subcutaneous placement of the cannula. After the cannula housing (not shown) is displaced at the infusion site, the needle hub 19 is removed from the cannula housing, exposing the barbs 13 and 14. The barbs 13 and 14 and undercuts 10 and 11 are adapted to mate and secure insertion device 18 when the needle hub 19 is re-inserted into the needle guard 17 without the cannula housing. The needle hub 19 is secured in the manner shown. The needle hub 19 is inside the opening 7 of the needle guard 17. In this covered position, the fingers 15 and 16 are extended beyond the hub holes 20 and 21 to the back end of the needle hub 19. In this configuration, the barbs 13 and 14 on the needle hub 19 are secured with the undercuts 10 and 11 in the needle guard 17.

The interaction between the barbs 13 and 14 and the undercuts 10 and 11 is more apparent in the partially transparent view shown in FIG. 5. When a cannula housing is removed, the barbs 13 and 14 on the needle hub 19 are exposed. The barbs 13 and 14 mate into the undercuts 10 and 11 in the needle guard 17 when the needle hub 19 is received into the body 2 of the needle guard 17. The barbs 13 and 14 and the undercuts 10 and 11 mate to prevent the needle hub 19 from slipping out of the needle guard 17. In this position, the needle guard 17 covers the needle 1 to prevent unintended injuries during disposal. In other alternatives, the number of undercuts and barbs may be varied. For example, one alternative locking structure may have one undercut on the needle guard mated to one barb on the needle hub. Additionally, the needle guard and needle hub may use alternative locking structures to the undercuts and barbs, such as such as hooks, clips, or other connecting pieces.

In the alternative covered position, shown in FIG. 5, the fingers 15 and 16 may extend past the hub holes 20 and 21 and avoid interaction with the hub holes 20 and 21. In an alternative embodiment, the needle guard may have top and bottom fingers with barbs. In this alternative, the securing of the needle hub inside the needle guard can be achieved by squeezing the needle guard so that the top finger barbs mate with the bottom finger barbs to secure the needle guard over the needle hub. The needle guard may be made from a suitable plastics material that will allow it to flex and bend, such as polypropylene. However, the needle guard may also be made out of a non-flexible material, such as polycarbonate, if preferred. Alternatively, the needle guard may be made out of any suitable flexible or non-flexible material such as polyethylene, polyurethane, polyvinyl chloride, resins, polymers, ceramics, composites, or the like.

Without being limited, several other embodiments of needle guards comprising a body and locking mechanism, and the corresponding insertion devices, are described below. Because the structure of each embodiment focuses on the interaction between the needle hub and the needle guard, each embodiment is described with respect to the needle hub and the needle guard. It is intended that the assembly not described have the same mating structure as the element that is described and is compatible with each of the embodiments.

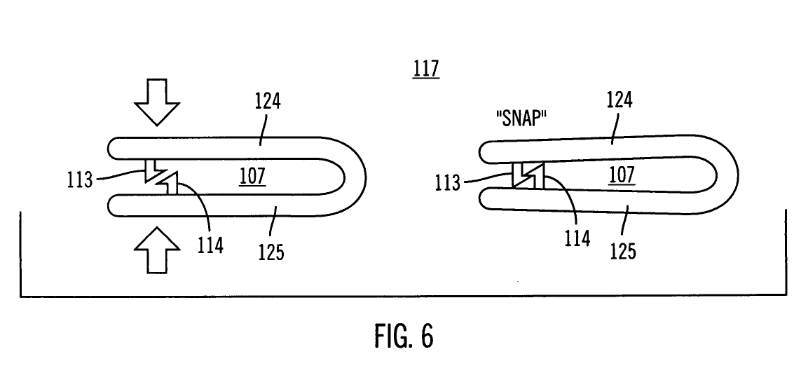

As shown in FIG. 6, in one embodiment the needle guard may have barbs 113 and 114 on the inner side, located at the top part 124 and bottom part 125 of the opening 107. In this alternative, the needle guard fits over the needle hub (not shown). The securing of the needle hub (not shown) inside the needle guard 117 can be achieved by using a needle guard that is longer and larger than the needle hub (not shown). By squeezing the needle guard 117 inwards the top barb 113 mates with the bottom barb 114 to secure the needle guard 117 over the needle hub to cover the needle (not shown). In an alternative, there may be a set of two barbs each on the top and bottom parts of the opening. The locking mechanism may hold the covered position either temporarily or permanently.

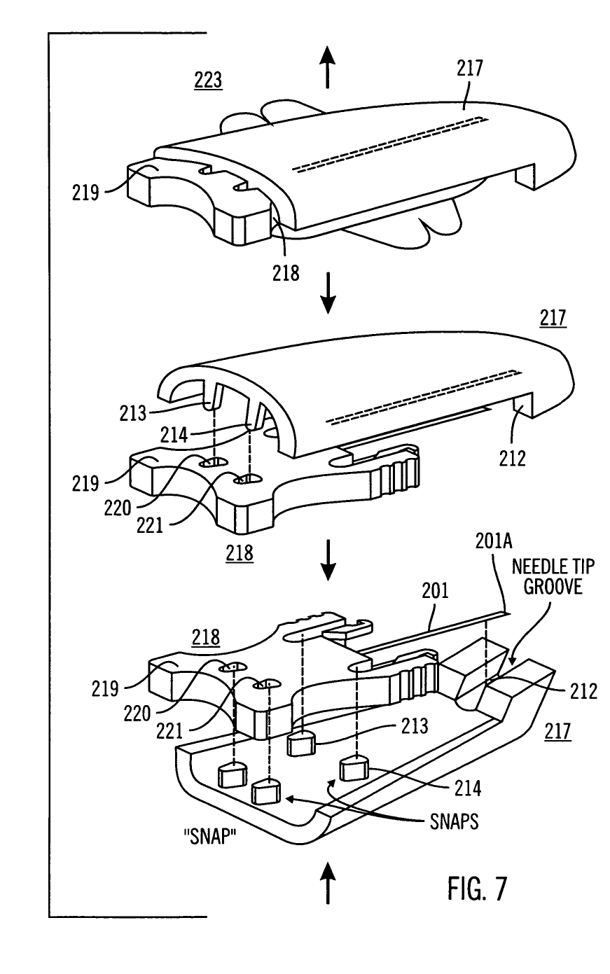

As shown in FIG. 7, in one embodiment the needle guard 217 is configured as one piece that fits over one side of the needle hub 219 of the assembly 223. The needle guard 217 can be removed by lifting it off the insertion device 218. A needle tip groove 212 is located at one end of the needle guard 217 where the needle tip 201A is held when the needle guard 217 is attached to the needle hub 219. The needle guard 217 includes barbs 213 and 214 that extend outwards and mate with corresponding hub holes 220 and 221 on the needle hub 219. The needle guard 217 may be snapped over the needle hub 219 to cover the needle 201. This position may be temporary or permanent. The tip 201A of the needle 201 is held inside the needle tip groove 212 when the needle guard 217 is connected to the needle hub 219.

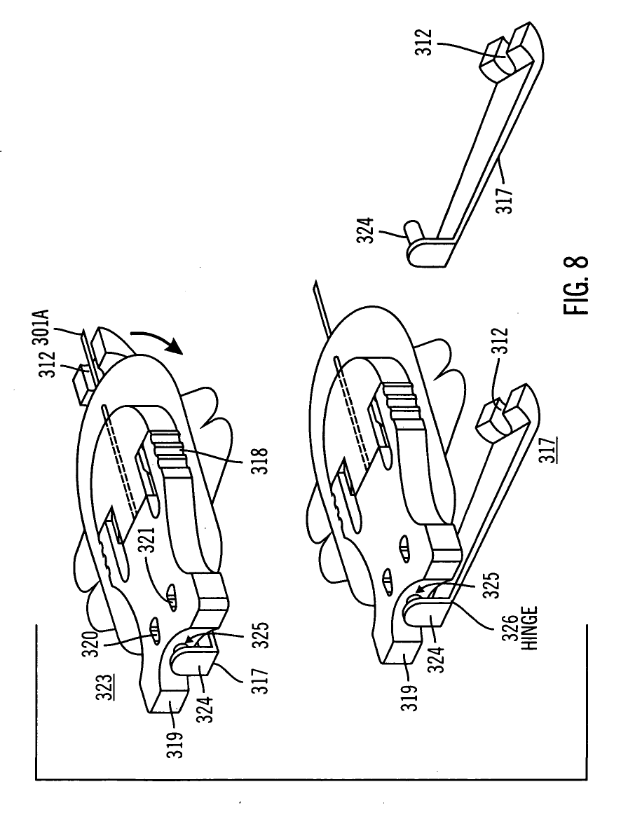

As shown in FIG. 8, in one embodiment the needle guard 317 is a long, narrow structure attached to the insertion device 318 at the back end of the needle hub 319. At the other end, the needle guard 317 includes a needle tip groove 312 wherein the needle 301 is held. The needle guard 317 may include an arm 324 that extends to mate into a rear hub hole 325 located at the back end of the needle hub 319. The needle guard 317 may also have barbs (not shown) to mate to the hub holes 320 and 321 on the needle hub 319. A joint part 326 is located on the needle guard 317 below the arm 324 and above the barbs. The joint part 326 is an area of high flexibility that allows the needle guard 317 to be removed by bending to disconnect the needle guard 317 from the hub holes 320 and 321 and bend the needle guard 317 away from the needle tip 301A. To cover the needle 301, the arm 324 and/or optional barbs (not shown) may be re-inserted into the corresponding hub holes 320 and 321 so that the needle tip 301A is held in the needle tip groove 312. The covering of the needle may be temporary or permanent.

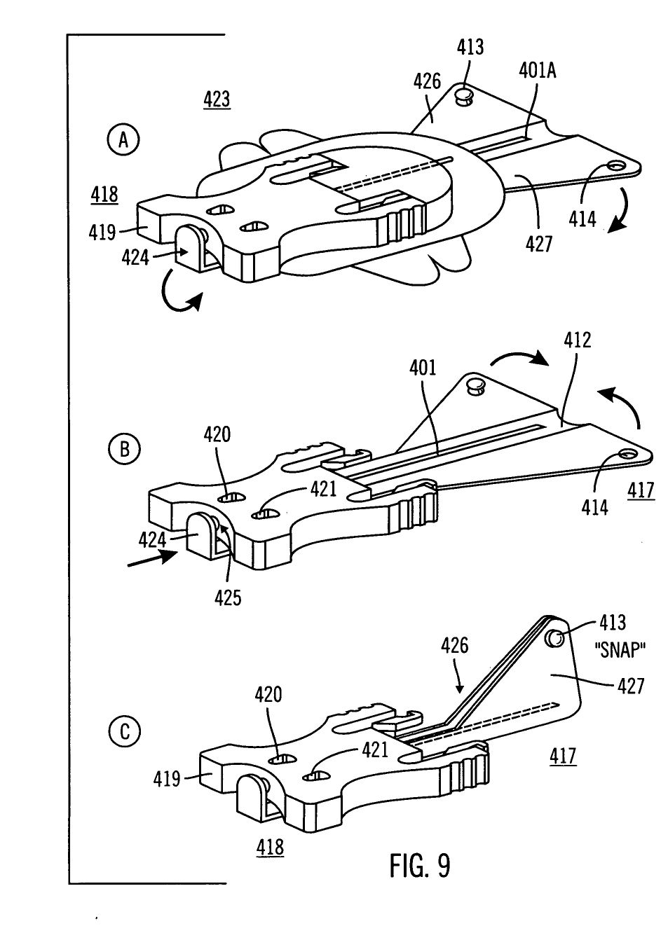

As shown in FIG. 9, in one embodiment the needle guard 417 is a structure attached to the insertion device 418 at the back end of the needle hub 419. At one end, the needle guard 417 is tabbed and includes a needle tip grove 412. The tabs 426 and 427 are thin, wide strips of material that can be folded together. One tab may have a barb 413 that snaps into a corresponding hole 414 on the second tab to secure the tabs 426 and 427 together. At the other end, the needle guard 417 may include an arm 424 that extends to mate into a rear hub hole 425 located at the back end of the needle hub 419. The insertion device 418 and the needle guard 417 may be received as an entire assembly 423. Before insertion, the needle guard 417 is removed from the needle hub 419 by bending the tabbed end downward and removing the arm 424 from the rear hub hole 425. The needle guard 417 may be replaced on the needle hub 419 by inserting the arm 424 of the needle guard 417 into the rear hub hole 425 and pivoting the tabbed end upwards. Once the needle hub 419 and the needle guard 417 are attached together, the needle 401 of the insertion device 418 lies in the needle tip grove 412. In this position, the tabs 426 and 427 can be folded upwards and snapped together to secure the needle tip 401A within the needle guard 417. This position may be temporary or permanent. The needle guard 417 may also have additional barbs (not shown) to mate to the hub holes 420 and 421 so that the needle tip 401A is further secured in the needle tip groove 412.

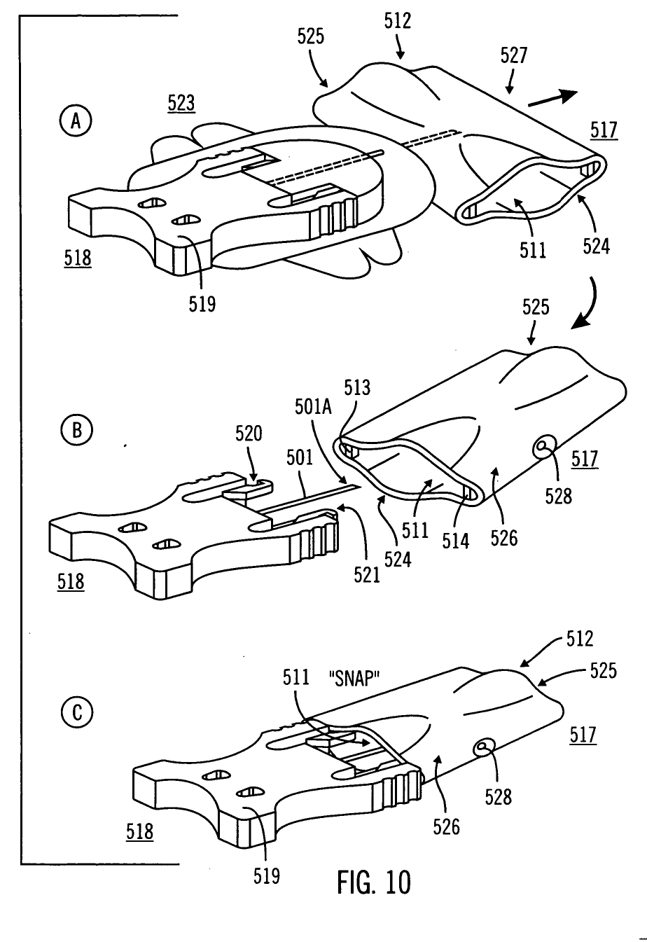

As shown in FIG. 10, in one embodiment the needle guard 517 is a symmetrical structure with four sides 524, 525, 526 and 527 and includes two cavities 511 and 512 that can be entered from two sides 524 and 525. There is a needle port 528 on the side 526 of the needle guard 517 that does not have the cavities 511 and 512. The needle 501 of the insertion device 518 can be inserted into the needle port 528 so that the needle tip 501A is not exposed. The needle guard 517 and insertion device 518 can be received as an entire assembly 523. In this configuration, the needle 501 is held within the needle port 528 on the side 526 of the needle guard 517. The needle guard 517 remains attached to the needle 501 by friction. Before insertion, the needle 501 is withdrawn from the needle port 528. Once removed, the needle guard 517 can be rotated so that the needle 501 can be re-inserted by the user into either of the cavities 511 and 512 through either of the two sides 524 and 525. When the needle 501 is entirely within one of the cavities 511 and 512, detents inside of the cavities 511 and 512 mate with corresponding catches on the needle hub 519. The detents 513 and 514 and the corresponding catches 520 and 521 inside cavity 511 are shown. In this position, which can be temporary or permanent, the needle 501 is secured within the needle guard 517. In an alternative, the needle guard may include needle ports on all four sides.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. An insertion set, comprising:
an insertion device including a needle hub and a needle attached to the needle hub,
a cannula housing removably attachable to the needle hub, and
a needle guard adapted to cover the needle of the needle hub, wherein the needle guard includes an opening adapted to receive the needle hub and locks adapted to secure the insertion device in the needle guard when the needle is covered by the needle guard.

2. The insertion set of claim 1, wherein the hub includes a back end and a hub hole in the back end.

3. The insertion set of claim 1, wherein the locks of the needle guard are undercuts protruding into the opening and wherein the needle hub includes barbs that are adapted to mate with the undercuts.

4. The insertion set of claim 1, wherein the needle guard further includes a finger that includes a finger barb, and wherein the finger barb is adapted to mate into a hub hole of the needle hub to secure the needle guard over the needle hub when connected to the cannula housing.

5. The insertion set of claim 4, wherein the finger extends from a side of the needle guard where the insertion device is received into the opening.

6. The insertion set of claim 4, wherein the finger barb is releasable from the hub hole to allow removal of the needle hub from the needle guard.

7. The insertion set of claim 1, wherein the needle guard is composed of a flexible material.

8. The insertion set of claim 1, wherein the needle guard is composed of a material selected from the group consisting of polypropylene and polycarbonate.

9. The insertion set of claim 1, wherein the needle guard is composed of a material selected from the group consisting of polyethylene, polyurethane, polyvinyl chloride, resins, polymers, ceramics, and composites.

10. A needle guard adapted to cover a needle of an insertion device,
wherein the needle guard includes an opening adapted to receive the insertion device and locks adapted to secure the insertion device in the needle guard when the needle is covered by the needle guard.

11. The needle guard of claim 10, wherein the locks are undercuts protruding into the opening and wherein the needle hub includes barbs that are adapted to mate with the undercuts.

12. The needle guard of claim 10 further including a finger that includes a finger barb that is adapted to mate to a back end of the needle hub when the needle hub is received by the needle guard, and wherein the needle hub includes a hub and a hole in the hub that is adapted to receive the finger barb, in a covered position.

13. The insertion set of claim 12, wherein the finger extends from a side of the needle guard where the insertion device is received into the opening.

14. The needle guard of claim 12, wherein the finger barb is releasable from the hub hole to allow removal of the insertion device from the needle guard.

15. The needle guard of claim 10 being composed of a flexible material.

16. The needle guard of claim 10 being composed of a material selected from the group consisting of polypropylene and polycarbonate.

17. The needle guard of claim 10 being composed of a material selected from the group consisting of polyethylene, polyurethane, polyvinyl chloride, resins, polymers, ceramics, and composites.

18. An insertion set, comprising:
an insertion device including a hub and a needle attached to the hub, and
a needle guard adapted to cover the needle of the insertion device, wherein the needle guard includes an opening adapted to receive the insertion device and locks adapted to secure the insertion device in the needle guard when the needle is covered by the needle guard.

19. The insertion set of claim 18, wherein the hub includes a back end and a hub hole in the back end.

20. The insertion set of claim 18, wherein the locks are undercuts protruding into the opening and wherein the needle hub includes barbs that are adapted to mate with the undercuts.

21. The insertion set of claim 18, wherein the needle guard further includes a finger that includes a finger barb, and wherein the finger barb is adapted to mate into a hub hole of the needle hub to secure the needle guard over the needle hub.

22. The insertion set of claim 21, wherein the finger extends from a side of the needle guard where the insertion device is received into the opening.

23. The insertion set of claim 21-, wherein the finger barb is releasable from the hub hole to allow removal of the insertion device from the needle guard.

24. The insertion set of claim 18, wherein the needle guard is composed of a flexible material.

25. The insertion set of claim 18, wherein the needle guard is composed of a material selected from the group consisting of polypropylene and polycarbonate.

26. The insertion set of claim 18, wherein the needle guard is composed of a material selected from the group consisting of polyethylene, polyurethane, polyvinyl chloride, resins, polymers, ceramics, and composites.

27. A method for covering an insertion needle, comprising:
inserting an insertion device into a needle guard, wherein the insertion device includes the needle and a hub and the needle guard includes an opening adapted to receive the insertion device and locks adapted to secure the insertion device in the needle guard when the needle is covered by the needle guard,
covering the needle with the needle guard; and
securing the insertion device within the needle guard, wherein the needle is covered by the needle guard.

28. The method of claim 27, wherein the hub includes a back end and a hub hole in the back end.

29. The method of claim 27, wherein the locks are undercuts protruding into the opening and wherein the needle hub includes barbs that are adapted to mate with the undercuts.

30. The method of claim 27, wherein the needle guard further includes a finger that includes a finger barb, and wherein the barb is adapted to mate into a hub hole of the needle hub to secure the needle guard over the needle hub.

31. The insertion set of claim 30, wherein the finger extends from a side of the needle guard where the insertion device is received into the opening.

32. The method of claim 30, wherein the finger barb is releasable from the hub hole to allow removal of the insertion device from the needle guard.

33. The method of claim 27, wherein the needle guard is composed of a flexible material.

34. The method of claim 27, wherein the needle guard is composed of a material selected from the group consisting of polypropylene and polycarbonate.

35. The method of claim 27, wherein the needle guard is composed of a material selected from the group consisting of polyethylene, polyurethane, polyvinyl chloride, resins, polymers, ceramics, and composites.
